(19) United States
(12) Patent Application Publication
(10) Pub. No.: US 2022/0023194 A1
(43) Pub. Date: Jan. 27, 2022

(54) Cosmetic Mask Comprising a Plant Paper and Humectant

(71) Applicant: SWM LUXEMBOURG, CONTERN (LU)

(72) Inventor: Cédric Rousseau, Le Mans (FR)

(21) Appl. No.: 17/296,693

(22) PCT Filed: Nov. 26, 2019
(86) PCT No.: PCT/EP2019/082598

§ 371 Date: May 25, 2021

(30) Foreign Application Priority Data
Nov. 26, 2018 (FR) 18 71869

(51) Int. Cl. A61K 8/9789; A61K 31/352; A61K 31/05; D21H 11/12; D21H 21/20; D21H 21/22; D21H 27/00

(52) U.S. Cl. CPC A61K 8/9789; A61K 31/352; A61K 31/05; D21H 27/00; D21H 21/20; D21H 21/22; D21H 11/12

(57) ABSTRACT

The invention relates to a cosmetic mask comprising a plant paper, a wet strength agent and a humectant.

The invention relates to a cosmetic mask comprising a plant paper, a wet strength agent and a humectant.

Cosmetic masks are commonly used to moisturize the skin, brighten the complexion, purify the face or firm the skin. Typically, the user applies the mask to his/her face for 10 to 20 minutes and the lotion contained therein is then released onto the face to obtain the desired cosmetic effect. The success of cosmetic masks is such that worldwide demand has risen sharply in recent years.

To satisfy users, a cosmetic mask must have several features. It must have a good sensory effect throughout its use, in particular it must be pleasant for the eye (glossy, natural) and for the skin (soft, silky, supple). Ideally, the mask has the following properties: it should also be pleasant to handle (easy to unfold and place, no loss of particles); it should have a good drape; the mask should be resistant to marking, i.e. when unfolded, the mask should not show fold marks; it should be conformable, i.e. adapt to the part of the body to which it is applied; the mask must also have a good reservoir effect, i.e. it must be able to absorb the cosmetic lotion containing the cosmetic active agents in order to release it to the skin. Moreover, the mask must also have wet strength properties to be resistant when moistened/impregnated with lotion.

Masks are traditionally made of paper or of synthetic fiber. The processes for producing conventional masks use hazardous chemicals such as soda, chlorine, chlorine dioxide, oxygen, ozone, hydrogen peroxide or hydrocarbons. These conventional masks can therefore be potentially dangerous for users. Moreover, these conventional masks are not very “natural”.

There is therefore user demand for less dangerous, more “natural” cosmetic masks. In order to meet this demand, US 2015/0056255 describes, for example, masks in which paper or synthetic fibers are partially substituted by plant fibers. The features of these masks, in particular the flexibility, the ease of use and the comfort, can be improved.

The inventors have developed a cosmetic mask comprising:
a plant paper comprising from 1% to 95% plant fibers and from 5% to 99% cellulose fibers by weight based on the total amount of fibers of said plant paper,
a wet strength agent, and
a humectant.

Advantageously, the humectant gives the mask according to the invention its conformability, softness, drape and resistance to marking. In addition, the mask according to the invention is advantageously capable of absorbing, retaining and releasing a cosmetic lotion in a satisfactory manner. The mask according to the invention is also simple to manufacture. The mask according to the invention is also biodegradable and compostable.

In addition, the mask according to the invention, has an odor and a color related to the plant raw materials used.

For the purposes of the present invention, “mask” means a flexible substrate having a shape to fit parts of the human body. Typically, the mask according to the invention is a facial mask, i.e. a mask with a shape to fit the characteristics of the face and having holes for the eyes, nose and mouth.

According to a particular embodiment, the amount of plant fibers in the plant paper can be from 2% to 85%, most particularly from 5% to 80% by weight based on the total amount of fibers in the plant paper.

Plant fibers are the fibrous parts of a plant.

Plant paper can be obtained from any type of plant, in particular mint, eucalyptus, rose, edelweiss, amaranth, lavender, grapevine, calendula, arnica, baobab, lemongrass, tea, ginger, chamomile, or a mixture thereof, in particular mint, rose, edelweiss, tea, chamomile, or a mixture thereof.

Typically, plant paper results from the whole plant, from a part of the plant or from a mixture of different parts of the plant. The part of the plant can typically be the flower, leaf, fruit, stem, root, or a mixture thereof. The plant part can also be the result of the mechanical, chemical or mechanochemical transformation of one or more parts of the plant, such as grape marc.

According to an embodiment, the plant paper of the mask according to the invention comprises plant fibers having undergone extraction in a solvent.

Typically, the amount of plant fibers having undergone extraction in a solvent in the plant paper is at least 5%, in particular from 10% to 90%, most particularly from 15% to 80% by weight based on the total amount of fibers of said plant paper.

Typically, the plant paper comprises less than 10% of solvent-soluble extract of the plant by weight of dry matter of said plant paper.

The solvent-soluble extract corresponds to all the non-fibrous parts of the plant that are soluble in the solvent.

Typically, the plant fibers having undergone extraction in a solvent according to the invention are obtained by an extraction and separation process. In such a process, one or more parts of the plant are mixed with the solvent, for example in an extractor, to extract the solvent-soluble extract (extraction step). The solvent-soluble extract is then separated from the fibrous parts, for example by passing through a screw press or a hydraulic press, to obtain, on the one hand, the plant fibers having undergone extraction in a solvent and, on the other hand, the solvent-soluble extract (separation step). In such an extraction and separation process, plant fibers having undergone extraction in a solvent are extracted and then separated from the solvent-soluble extract.

The plant fibers having undergone extraction in a solvent according to the invention may therefore include a residual fraction of the solvent-soluble extract, which explains why the plant paper comprises less than 10% solvent-soluble extract of the plant by weight of dry matter of the plant paper. Typically, the plant paper comprises less than 5%, less than 4%, less than 3%, less than 2%, less than 1% solvent-soluble extract of the plant by weight of dry matter of the plant paper. According to a particular embodiment, the plant paper contains no extract of the plant soluble in the solvent.

The following technique will be used to determine the percentage of solvent-soluble extract by weight of dry matter in the plant paper. The plant paper to be analyzed is ground to a particle size of 2 mm or less. The ground plant paper is then mixed with boiling water for 10 minutes to extract the solvent-soluble extract. The weight of dry matter in the plant paper of solvent-soluble extract is calculated by the difference between the dry weight of the plant paper sample and the dry weight of the fibrous residue after extraction.

The extraction and separation process makes it possible to achieve little or no solvent-soluble extract in the plant paper according to the invention. Without wishing to be bound by any theory, the inventors are of the opinion that extraction in solvent gives the mask according to the invention its mechanical and sensory properties, in particular an odor and color related to the plant raw materials used.

The extraction step carried out to obtain the plant fibers having undergone extraction in a solvent is a gentle method that does not use compounds such as soda or organic solvents that are hazardous to humans and the environment. It is also different from the delignification treatments used for paper production and bleaching treatments. Typically, delignification treatments are those known as mechanical pulp, thermo-mechanical pulp, chemical-mechanical pulp or chemical pulp. Typically, bleaching treatments are those using chlorine, chlorine dioxide, oxygen, ozone or hydrogen peroxide. Advantageously, solvent extraction according to the invention does not require the use of the hazardous chemicals listed above. Therefore, the mask according to the invention comprising a plant paper comprising plant fibers having undergone extraction in a solvent is less dangerous for the user than a mask made of paper. In addition, solvent extraction preserves the plant's active agents, which can therefore be released for the benefit of the user of the mask according to the invention comprising a plant paper comprising plant fibers having undergone extraction in a solvent.

According to a particular embodiment, the solvent is an aqueous solvent, most particularly the solvent is water.

Typically, the aqueous solvent can be a 70-30 (v/v) water-alcohol mixture.

According to the embodiment wherein the solvent is water, the extraction is carried out at atmospheric pressure and the temperature of the water is from 40° C. to 100° C., in particular from 60° C. to 90° C., more particularly from 70° C. to 80° C.

According to another embodiment, the plant paper of the mask according to the invention comprises plant fibers not having undergone extraction in a solvent.

The amount of plant fibers not having undergone extraction in the solvent in the plant paper according to the invention is at most 25% by weight based on the total amount of fibers of said plant paper. Typically, the amount of fibers not having undergone extraction in the solvent is from 1% to 22% by weight, in particular from 5 to 20%.

Typically, plant fibers not having undergone extraction in a solvent are present in the plant paper of the mask according to the invention in the form of particles of the whole plant, a part of the plant or a mixture of different parts of the plant, particularly in the form of particles of flower or of leaf of the plant.

Typically, these particles have a size of less than 5 mm, in particular from 0.5 mm to 4 mm, most particularly from 1 mm to 3 mm.

According to another embodiment, the plant paper according to the invention comprises plant fibers not having undergone extraction in a solvent and plant fibers having undergone extraction in a solvent.

Typically, the amount of cellulose fibers in the plant paper according to the invention is from 10% to 98%, in particular from 20% to 95% by weight based on the total amount of fibers in the plant paper.

For the purposes of the present invention, “cellulose fibers” refers to Tencel® fibers (cellulose fiber ground and dissolved in N-methylmorpholine N-oxide monohydrate), viscose fibers (obtained by dissolution of cellulose by modification of its hydroxyl groups with carbon disulfide (CS2), then its precipitation in the presence of sulfuric acid (H2SO4) in order to obtain yarns), fiber from wood pulp, hemp, bamboo, cotton, kapok, coconut, flax, ramie, jute, sisal, kenaf, abaca, sisal, raffia, papyrus, reed, wheat, sugar cane, corn, sorghum, or a mixture thereof.

Typically, the wood pulp is hardwood pulp, softwood pulp, or their mixture.

Typically, the cellulose fibers from wood pulp, hemp, bamboo, cotton, kapok, coconut, flax, ramie, jute, sisal, kenaf, abaca, sisal, raffia, papyrus, reed, wheat, sugar cane, corn, sorghum, or their mixture are refined and have a Schopper-Riegler degree (° SR) of 15° SR to 90° SR, in particular of 30° SR to 75° SR, most particularly of 50° SR to 65° SR.

Typically, the plant fibers, in particular the plant fibers having undergone extraction in a solvent, are also refined and have a Schopper-Riegler degree (° SR) of 15° SR to 90° SR, in particular of 30° SR to 75° SR, most particularly of 50° SR to 65° SR.

Cellulose fibers can be added to plant paper to modify the properties of the plant paper. Typically, the properties of the plant paper that can be modified are mechanical resistance properties such as tensile strength, tearing, bursting, folding or bending resistance, optical properties such as whiteness, opacity or gloss, or texture properties such as basis weight, porosity or permeability to air or a liquid.

According to a specific embodiment, the plant paper of the mask according to the invention comprises plant fibers having undergone extraction in a solvent, plant fibers not having undergone extraction in a solvent, and cellulose fibers, wherein:
the amount of plant fibers having undergone extraction in a solvent is from 10% to 20% by weight based on the total amount of fibers of said plant paper,
the amount of plant fibers not having undergone extraction in the solvent is from 1% to 10% by weight based on the total amount of fibers of said plant paper, and
the amount of the cellulose fibers is from 70% to 89% by weight based on the total amount of fibers of the plant paper, the sum of the amounts of the plant fibers having undergone extraction in a solvent, the plant fibers not having undergone extraction in the solvent and the cellulose fibers being 100% by weight based on the total amount of fibers of the plant paper.

According to another specific embodiment, the plant paper of the mask according to the invention comprises plant fibers having undergone extraction in a solvent and cellulose fibers, wherein:
the amount of plant fibers having undergone extraction in a solvent is from 60% to 80% by weight based on the total amount of fibers of said plant paper, and
the amount of the cellulose fibers is from 20% to 40% by weight based on the total amount of fibers of the plant paper,
   the sum of the amounts of the plant fibers having undergone extraction in a solvent and the cellulose fibers being 100% by weight based on the total amount of fibers of the plant paper.

According to another specific embodiment, the plant paper of the mask according to the invention comprises plant fibers not having undergone extraction in a solvent and cellulose fibers, wherein:
the amount of plant fibers that have not undergone extraction is from 5% to 20% by weight based on the total amount of fibers of said plant paper, and
the amount of the cellulose fibers is from 80% to 95% by weight based on the total amount of fibers of the plant paper,
   the sum of the amounts of the plant fibers that have not undergone extraction and the cellulose fibers being 100% by weight based on the total amount of fibers of the plant paper.

The mask according to the invention also comprises a humectant.

Advantageously, the humectant gives the mask according to the invention its conformability, softness, drape and resistance to marking. In addition, the mask according to the invention is advantageously capable of absorbing, retaining and releasing a cosmetic lotion in a satisfactory manner.

Typically, the humectant may be a sugar alcohol such as glycerin or sorbitol; a glycol, such as propylene glycol, butylene glycol, pentylene glycol or dipropylene glycol; or polyethylene glycol; or a mixture thereof.

According to a particular embodiment, the humectant is glycerin.

Typically, the amount of humectant in the mask according to the invention is less than 50%, in particular from 5% to 45%, more particularly from 7% to 40%, even more particularly from 10% to 30% by dry weight based on the dry weight of the plant paper of the mask according to the invention.

The mask according to the invention also comprises a wet strength agent.

A wet strength agent reduces the potential degradation of the mask if it is brought into contact with a liquid, such as water or a cosmetic lotion.

Typically, the wet strength agent can be selected from polyam ides, such as an epichlorohydrin resin, a polyamine-epichlorohydrin resin, a polyamide-epichlorohydrin resin, a poly(aminoamide)-epichlorohydrin resin, a urea-formaldehyde resin, a melamine-formaldehyde resin, an alkyl-ketene dimer, alkyl-succinic anhydride, a polyvinylamine, an oxidized polysaccharide and their mixture.

Typically, the amount of wet strength agent in the mask according to the invention is from 0.1% to 5%, in particular from 0.5% to 4%, more particularly from 1% to 3%, even more particularly from 1.5% to 2.5% by dry weight based on the dry weight of the cellulose fibers of the plant paper of the mask according to the invention.

Typically, the mask according to the invention may comprise, in addition to the plant paper according to the invention, an active principle selected from sebum regulators, antimicrobials, antibacterials, mattifiers, astringents, acidifiers, healing agents, exfoliants or keratoregulators, occlusives, protectors, disinfectants, emollients, nourishing agents, moisturizers, anti-aging agents, soothing agents, decongestants or veinotonics, occlusives, UV filters, hygroscopics, gelling agents, exfoliants, anti-free radical agents, cell regenerators or stimulants, firming agents, tensors, antiglycation agents, lightening agents, a cannabinoid such as cannabidiol (CBD) and tetrahydrocannabinol (THC) and mixtures thereof.

According to one embodiment of the invention, the active principle may be a cannabinoid such as cannabidiol (CBD) and tetrahydrocannabinol (THC) or a mixture thereof, in particular cannabidiol (CBD).

The cosmetic mask according to the invention is manufactured according to a process comprising the following steps:
a) mixing plant fibers and cellulose fibers to obtain a fiber mixture,
b) producing a plant paper from the fiber mixture using a wetlaid process,
c) cutting the plant paper produced and shaping to produce the mask, wherein the humectant and the wet strength agent are added before, during or after step b).

When the plant paper of the mask according to the invention comprises plant fibers having undergone extraction in a solvent, then the process comprises, before step a), steps of extracting and separating the plant fibers in a solvent to obtain the plant fibers having undergone extraction in a solvent. The extraction and separation steps are as described above in the section on the cosmetic mask.

When the plant paper of the mask according to the invention comprises plant fibers not having undergone extraction in a solvent, then the process comprises, before step a), a step of cutting the entire plant, a part of the plant or a mixture of different parts of the plant to obtain the particles including the plant fibers not having undergone extraction in a solvent. These particles are then mixed with cellulose fibers to obtain the fiber mixture.

When the cellulose fibers are refined, then the process comprises, before step a), a step of refining the cellulose fibers. The refined cellulose fibers are then mixed with the plant fibers in step a) to obtain the fiber mixture.

When the plant fibers are refined, then the process comprises, before step a), a step of refining the plant fibers. The refined plant fibers are then mixed with the cellulose fibers in step a) to obtain the fiber mixture.

When the plant fibers having undergone extraction in a solvent are refined, then the process comprises, after the extraction and separation steps and before step a), a step of refining the plant fibers. The refined plant fibers are then mixed with the cellulose fibers in step a) to obtain the fiber mixture.

When the cellulose fibers and the plant fibers, in particular the plant fibers having undergone extraction in a solvent, are refined, then the fiber mixture obtained in step a) can be refined before step b); or the cellulose fibers and the plant fibers can be refined separately before step a) as described above, and then the refined fibers can be mixed in step a) to obtain the fiber mixture.

The skilled person will be able to adapt the refining process to obtain refined fibers with the desired Schopper-Riegler degree.

Step b) involves a conventional wet paper production process, in particular a wet paper production process involving a dewatering screen, typically an inclined and rising dewatering screen or flat dewatering screen. The skilled person will be able to adapt the parameters of the wet process involving a dewatering screen to produce the plant paper according to the invention.

Typically, the wet strength agent can be added to the fiber mixture before it undergoes step b) or is added to the plant paper produced in step b) using a size press, coating or spraying. In particular, the wet strength agent may be added to the fiber mixture before it undergoes step b) to improve the interaction between the wet strength agent and the cellulose fibers.

Typically, the humectant can be added to the fiber mixture before it undergoes step b) or is added to the plant paper produced in step b) using a size press, coating or spraying. In particular, the humectant can be added is added to the plant paper produced in step b).

Typically, after step b), the plant paper of the mask according to the invention can be dried by a drying device, such as drying rollers or a tunnel.

The plant paper of the mask according to the invention can also undergo additional treatments known to the paper industry. Typically, one of these treatments allows the production of a multi-layer plant paper using multiple head boxes.

Step c) is a conventional cutting and shaping step to produce the mask.

### EXAMPLES

### Example 1

### Facial Mask Comprising a Plant Paper, a Wet Strength Agent and a Humectant

### Example 1.1

### The Plant is the Tea Plant

In this example, the tea plant fibers have undergone extraction in water.

Green tea leaves and water are mixed in a leaf-to-water mass ratio of 1:5. The mixture is heated to 85° C. for 20 minutes. This mixture then undergoes a separation step in a hydraulic press to separate the tea tree fibers having undergone extraction in water from the soluble extract.

The tea tree fibers having undergone extraction in water are then mixed with cellulose fibers from abaca and cellulose fibers from hardwood pulp to obtain a fiber mixture. The fibers in the fiber mixture are then refined to a Schopper-Riegler degree of 54° SR. Kymene GHP20 (a wet strength agent) is then added to the refined fiber mixture. This last mixture then passes over a dewatering screen to obtain a plant paper comprising 60% tea tree fibers having undergone extraction in water, 30% cellulose fibers from abaca and 10% cellulose fibers from hardwood pulp by weight based on the total amount of fibers in the plant paper. The plant paper also contains 2% Kymene GHP20 by dry weight based on the dry weight of the cellulose fibers.

Glycerin is then added to the plant paper using a size press. The amount of glycerin in the plant paper is 10% by dry weight based on the dry weight of the plant paper of the mask.

The plant paper is then cut and shaped to produce the facial mask.

### Example 1.2

### The Plants are Tea and Mint

In this example, tea tree fibers and mint fibers have undergone extraction in water.

Green tea leaves, mint leaves and water are mixed in a leaf-to-water mass ratio of 1:5. The mixture is heated to 85° C. for 20 minutes. This mixture then undergoes a separation step in a hydraulic press to separate the tea tree fibers and the mint fibers having undergone extraction in water from the soluble extracts.

The tea tree fibers and mint fibers having undergone extraction in water are then mixed with cellulose fibers from abaca to obtain a fiber mixture. The fibers in the fiber mixture are then refined to a Schopper-Riegler degree of 55° SR to 60° SR. Kymene GHP20 (a wet strength agent) is then added to the refined fiber mixture. This last mixture then passes over a dewatering screen to obtain a plant paper comprising 40% tea tree fibers having undergone extraction in water, 40% mint fibers having undergone extraction in water and 20% cellulose fibers by weight based on the total amount of fibers in the plant paper. The plant paper also contains 2% Kymene GHP20 by dry weight based on the dry weight of the cellulose fibers.

Glycerin is then added to the plant paper using a size press. The amount of glycerin in the plant paper is 10% by dry weight based on the dry weight of the plant paper of the mask.

The plant paper is then cut and shaped to produce the facial mask.

### Example 1.3

### The Plants are Tea Plants and Mint

The plant paper of Example 1.3 is obtained in a similar way to the plant paper of Example 1.2. The difference is that the amount of glycerin in the plant paper of Example 1.3 is 30% by dry weight based on the dry weight of the plant paper in the mask.

The plant paper of Example 1.3 is then cut and shaped to produce the facial mask.

### Example 1.4

### The Plant is the Rose

In this example, the rose fibers are derived from rose petals and have not undergone extraction in a solvent.

Refined cellulose fibers from abaca with a Schopper-Riegler degree of 52° SR are mixed with Kymene GHP20 (a wet strength agent). Rose petals cut into 2 mm particles are added to this mixture just before it passes over a dewatering screen to obtain a plant paper comprising 20% rose fibers not having undergone extraction and 80% cellulose fibers from abaca by weight based on the total amount of fibers in the plant paper. The plant paper also contains 2% Kymene GHP20 by dry weight based on the dry weight of the cellulose fibers.

Glycerin is then added to the plant paper using a size press. The amount of glycerin in the plant paper is 20% by dry weight based on the dry weight of the plant paper of the mask.

The plant paper is then cut and shaped to produce the facial mask.

### Example 1.5

### The Plant is Edelweiss

In this example, the edelweiss fibers are derived from edelweiss flowers and have not undergone extraction in a solvent.

Cellulose fibers from abaca are mixed with cellulose fibers from softwood pulp to obtain a mixture of cellulose fibers. The fibers of this mixture are refined and have a Schopper-Riegler degree of 54° SR. The refined fiber mixture is mixed with Kymene GHP20 (a wet strength agent). Edelweiss flowers cut into 2 mm particles are added to this mixture just before it passes over a dewatering screen to obtain the plant paper comprising 5% edelweiss fibers not having undergone extraction and 80% cellulose fibers from softwood pulp and 15% cellulose fiber from abaca by weight based on the total amount of fibers in the plant paper. The plant paper also contains 2% Kymene GHP20 by dry weight based on the dry weight of the cellulose fibers.

Glycerin is then added to the plant paper using a size press. The amount of glycerin in the plant paper is 20% by dry weight based on the dry weight of the plant paper of the mask.

The plant paper is then cut and shaped to produce the facial mask.

### Example 1.6

### The Plant is Edelweiss

The plant paper of Example 1.6 is obtained in a similar way to the plant paper of Example 1.5. The difference is that the cellulose fibers come only from abaca and the fibers in the mixture are refined and have a Schopper-Riegler degree of 53° SR.

The plant paper of Example 1.6 is then cut and shaped to produce the facial mask.

### Example 1.7

### The Plants are Chamomile and Rose

In this example, the chamomile fibers have undergone extraction in water, and the rose fibers were derived from rose petals and did not undergo extraction with water.

Whole chamomile plants and water are mixed in a plants-to-water mass ratio of 1:5. The mixture is heated to 85° C. for 20 minutes. This mixture then undergoes a separation step in a hydraulic press to separate the chamomile fibers having undergone extraction in water from the soluble extract.

The chamomile fibers having undergone extraction in water are then mixed with cellulose fibers from abaca and cellulose fibers from flax to obtain a fiber mixture. The fibers in the fiber mixture are then refined to a Schopper-Riegler degree of 55° SR to 60° SR. Kymene GHP20 (a wet strength agent) is then added to the refined fiber mixture. Rose petals are cut into 2 mm particles and added to this last mixture just before it passes over a dewatering screen to obtain a plant paper comprising 15% chamomile fibers having undergone extraction in water, 5% rose fibers, 65% cellulose fibers from flax and 20% cellulose fibers from abaca by weight based on the total amount of fibers in the plant paper. The plant paper also contains 2% Kymene GHP20 by dry weight based on the dry weight of the cellulose fibers.

Glycerin is then added to the plant paper using a size press. The amount of glycerin in the plant paper is 10% by dry weight based on the dry weight of the plant paper of the mask.

The plant paper is then cut and shaped to produce the facial mask.

### Example 1.8

### The Plants are Chamomile and Rose

The plant paper of Example 1.8 is obtained in a similar way to the plant paper of Example 1.7. The difference is that the amount of glycerin in the plant paper is 30% by dry weight based on the dry weight of the plant paper of the mask.

The plant paper of Example 1.8 is then cut and shaped to produce the facial mask.

### Comparative Example 1

### Facial Mask Comprising a Plant Paper, a Wet Strength Agent but Without Humectant

### Comparative Example 1.1

### The Plant is the Tea Plant

The plant paper of Comparative Example 1.1 is obtained in a similar way to the plant paper of Example 1.1, but it does not contain glycerin.

The plant paper of Comparative Example 1.1 is then cut and shaped to produce a glycerin-free facial mask.

### Comparative Example 1.2

### The Plants are Tea and Mint

The plant paper of Comparative Example 1.2 is obtained in a similar way to the plant paper of Example 1.2, but it does not contain glycerin.

The plant paper of Comparative Example 1.2 is then cut and shaped to produce a glycerin-free facial mask.

### Comparative Example 1.3

### The Plant is the Rose

The plant paper of Comparative Example 1.3 is obtained in a similar way to the plant paper of Example 1.4, but it does not contain glycerin.

The plant paper of Comparative Example 1.3 is then cut and shaped to produce a glycerin-free facial mask.

### Comparative Example 1.4

### The Plant is Edelweiss

The plant paper of Comparative Example 1.4 is obtained in a similar way to the plant paper of Example 1.5, but it does not contain glycerin.

The plant paper of Comparative Example 1.4 is then cut and shaped to produce a glycerin-free facial mask.

### Comparative Example 1.5

### The Plant is Edelweiss

The plant paper of Comparative Example 1.5 is obtained in a similar way to the plant paper of Example 1.6, but it does not contain glycerin.

The plant paper of Comparative Example 1.5 is then cut and shaped to produce a glycerin-free facial mask.

### Comparative Example 1.6

### The Plants are Chamomile and Rose

The plant paper of Comparative Example 1.6 is obtained in a similar way to the plant paper of Example 1.7, but it does not contain glycerin.

The plant paper of Comparative Example 1.6 is then cut and shaped to produce a glycerin-free facial mask.

### Example 2

### Sensory Evaluation of the Facial Masks of Example 1 and of Comparative Example 1

The facial masks of Example 1 and of Comparative Example 1 are impregnated with a cosmetic lotion.

The sensory properties of the impregnated facial masks of Example 1 and of Comparative Example 1 are evaluated by cosmetic panels that are organized with several panelists.

For each facial mask, each panelist considers different criteria, namely: dry softness, drape, folding, pleat marking, unfolding, conformability and moisture retention.

### Example 2-1

### Facial Masks of Example 1.1 and of Comparative Example 1.1

The panelists find the conformability of the facial mask of Example 1.1 to be better than that of the facial mask of Comparative Example 1.1. In addition, the facial mask of Example 1.1 retains moisture better and has fewer wrinkle marks than the facial mask of Comparative Example 1.1.

Thanks to the addition of glycerin, the facial mask of Example 1.1 is more satisfying than the facial mask of Comparative Example 1.1. In addition, thanks to its moisture retention capacity, it can easily retain the absorbed cosmetic lotion when not in use.

### Example 2-2

### Plant Papers of Examples 1.2 and 1.3 and of Comparative Example 1.2

The addition of glycerin greatly increases the softness and drape of the plant paper. The conformability and retention capacity of this plant paper is also improved by glycerin.

The facial masks of Examples 1.2 and 1.3 are more satisfactory than the facial mask of Comparative Example 1.2.

### Example 2-3

### Plant papers of Example 1.4 and of Comparative Example 1.3

All criteria are improved by the addition of glycerin, except for the folding/unfolding criteria, which remain constant. In particular, the panelists find the drape of the facial mask of Example 1.4 to be prettier than that of Comparative

Example 1.3 and the facial mask of Example 1.4 to retain moisture better.

Thanks to the addition of glycerin, the facial mask of Example 1.4 is more satisfying than the facial mask of Comparative Example 1.3. In addition, thanks to its moisture retention capacity, it can easily retain the absorbed cosmetic lotion when not in use.

### Example 2-4

### Plant papers of Examples 1.5 and 1.6 and of Comparative Examples 1.4 and 1.5

The addition of glycerin improves dry softness, drape and moisture retention, while folding and conformability remain constant. In particular, the panelists find the drape of the facial masks of Examples 1.5 and 1.6 to be prettier than that of the facial masks of Comparative Examples 1.4 and 1.5.

The facial masks of Examples 1.5 and 1.6 are therefore quite satisfactory as cosmetic facial masks. In addition, thanks to their moisture retention capacity, they can retain the absorbed cosmetic lotion until it is used.

### Example 2-5

### Plant papers of Examples 1.7 and 1.8 and of Comparative Example 1.6

The addition of glycerin greatly increases the softness, drape and resistance to marking of the plant paper containing chamomile fibers having undergone extraction in water and rose fibers not having undergone extraction in water. This is especially true for the facial mask of Example 1.8 containing 30% glycerin.

The facial masks of Examples 1.7 and 1.8 are therefore quite satisfactory as cosmetic facial masks.

1. Cosmetic mask comprising:
a plant paper comprising from 1% to 95% plant fibers and from 5% to 99% cellulose fibers by weight based on the total amount of fibers of said plant 5 paper,
a wet strength agent, and
a humectant.

2. Mask according to claim 1 wherein the plant is selected from mint, eucalyptus, rose, edelweiss, amaranth, lavender, vine, calendula, arnica, baobab, lemongrass, tea, ginger, chamomile, or a mixture thereof.

3. Mask according to claim 1 wherein the plant paper comprises plant fibers having undergone an extraction in a solvent.

4. Mask according to claim 3 wherein the amount of plant fibers having undergone extraction in a solvent in the plant paper is at least 5% by weight based on the total amount of fibers of said plant paper.

5. Mask according to claim 1 wherein the plant paper comprises plant fibers not having undergone an extraction in a solvent.

6. Mask according to claim 5 wherein the amount of plant fibers not having undergone extraction in the solvent in the plant paper is at most 25% by weight based on the total amount of fibers of said plant paper.

7. Mask according to claim 1 wherein the amount of humectant is from 5% to 45%, by dry weight based on the dry weight of the plant paper of the mask.

8. Mask according to claim 1 wherein the amount of wet strength agent in the plant paper according to the invention is from 0.1% to 5%, by dry weight based on the dry weight of the cellulose fibers of the plant paper of the mask.

9. Mask according to claim 1 further comprising an active principle selected from sebum regulators, antimicrobials, antibacterials, mattifiers, astringents, acidifiers, healing agents, exfoliants or keratoregulators, occlusives, protectors, disinfectants, emollients, nourishing agents, moisturizers, anti-aging agents, soothing agents, decongestants or veinotonics, occlusives, UV filters, hygroscopics, gelling agents, exfoliants, anti-free radical agents, cell regenerators or stimulants, firming agents, tensors, antiglycation agents, lightening agents, a cannabinoid and mixtures thereof.

10. Mask according to claim 9 wherein the active principle is a cannabinoid selected from cannabidiol, tetrahydrocannabinol and mixtures thereof.

11. Mask according to claim 1 which is a facial

12. Process for manufacturing a mask as defined in claim 1 comprising the following steps:
a. mixing plant fibers and cellulose fibers to obtain a fiber mixture,
b. producing a plant paper from the fiber mixture using a wetlaid process,
c. cutting the plant paper produced and shaping to produce the mask, wherein the humectant and the wet strength agent are added before, during or after step b).

13. Process according to claim 12 wherein the humectant is added to the plant paper produced in step b) by means of a size press, coating or spraying.

14. Process according to claim 12 comprising, before step a), steps of extracting and separating plant fibers in a solvent to obtain plant fibers having undergone an extraction in a solvent.
